(19) United States
(12) Patent Application Publication
(10) Pub. No.: US 2008/0295186 A1
(43) Pub. Date: Nov. 27, 2008

(54) Neurotrypsin overexpressing animal

(76) Inventors: Peter Sonderegger, Zurich (CH); Jan Willem Vrijbloed, Schlieren (CH)

Correspondence Address: BINGHAM MCCUTCHEN LLP, 2020 K Street, N.W., Intellectual Property Department, WASHINGTON, DC, 20006, US

(21) Appl. No.: 12/007,928

(22) Filed: Jan. 17, 2008

Related U.S. Application Data
(63) Continuation-in-part of application No. 10/843,299, filed on May 12, 2004.
Application No. 10/843,299 is a continuation-in-part of application No. 09/403,724, filed on Dec. 20, 1999, now abandoned.

(51) Int. Cl. A01K 67/027; C12N 5/06; C12N 15/00; C12N 15/63

(52) U.S. Cl. 800/3; 800/13; 800/14; 800/18; 435/354; 435/320.1; 800/25; 800/22

(57) ABSTRACT

The invention describes a transgenic non-human animal overexpressing neurotrypsin wherein the animal exhibits symptoms of sarcopenia.

### CROSS REFERENCE TO RELATED APPLICATIONS

This is a CIP of Ser. No. 10/843,299, filed May 12, 2004, which is a CIP of Ser. No. 09/403,724, filed Dec. 20, 1999, now abandoned. The disclosures of these applications are incorporated herein by reference in their entirety.

### TECHNICAL FIELD

The present invention relates to the field of sarcopenia and animal models, screening systems and genetic constructs for the study thereof.

### BACKGROUND ART

Sarcopenia plays an important role in the pathogenesis of frailty and functional impairment that occurs with old age. The disease is characterized by a loss of contractile muscular strength, decreased metabolic rate, gradual reduction of bone density and decreased aerobic capacity. Several independent studies demonstrate that neural input to some muscle fibers is disrupted with age, resulting in subsequent atrophy and eventually the disappearance of the denervated fibers. Muscle fibers that do not receive nerve input become progressively atrophic within weeks and eventually disappear (Vandervoort, A. A. Muscle 8. Nerve 25: 17-25, 2002; Kamel. H K, Nutrition Reviews 61: 157-167, 2003).

Deterioration of the neuromuscular junction, ultimately causing structural and functional denervation, followed by atrophy and ultimately loss of denervated muscle fibers, contributes significantly to sarcopenia (Luff, A R., Ann, t,tY. Acad. Sci. 854: 92.101, 1998). Factors causing sarcopenia include the loss of motorneurons, the remodeling at the NMJ that include loss of pre- and postsynaptic sites as well as decreased physical activity, altered hormonal status, inflammatory mediators, and altered protein synthesis.

Histological analysis of muscle fibers from persons suffering of sarcopenia show a reduced number of muscle fibers, a greater heterogeneity of fiber size, accumulation of internal nuclei in muscle fibers, denervation of muscle fibers, fiber type grouping and infiltration of fat and connective tissue.

In order to correct this condition, it is necessary to increase the transmission between nerve and muscle cells. Traditionally this can be done by physical activity, yet at a certain age, this cannot fully compensate for the loss in muscle mass. It is therefore desirable to induce increased transmission through medication. In order to study sarcopenia and the effectiveness of potential pharmaceutical compounds, animal models for sarcopenia are desirable, which, up to now, were not available since animals usually do not reach an age where they develop age-related sarcopenia.

### DISCLOSURE OF THE INVENTION

It is therefore a general object of the invention to provide a transgenic non-human animal which allows the study of sarcopenia as well as the identification and validation of candidate compounds that alleviate, prevent or delay the progression of sarcopenia.

It has surprisingly been found that neurotrypsin (NT) overexpressing mice show an early onset of sarcopenia. The model organism of the present invention is useful for the study of sarcopenia, for analyzing the physiological function of NT in vivo as well as the identification and validation of the therapeutic effect of candidate compounds in the treatment of sarcopenia.

Systematic analysis of muscles of transgenic mice that overexpress neurotrypsin in motoneurons reveal a striking atrophy of skeletal muscles with a debilitating reduction of muscle strength. Quantitative studies on muscle cross-sections of adult transgenic mice revealed a reduction of the number of muscle fibers ranging from 18 to 48% depending on the level of neurotrypsin overexpression. This atrophy-causing effect strictly depends on the proteolytic activity of neurotrypsin, since muscles of mice overexpressing a catalytically inactive form of neurotrypsin exhibited normal fiber numbers.

Experimental data indicate that NT is causally related to endplate deterioration. The NT-dependent deterioration of the neuromuscular junction and associated muscle atrophy in transgenic mice are in accordance with the observations made at muscles and neuromuscular junctions of humans with age-dependent skeletal muscle atrophy. Therefore, the inhibition of NT is expected to have a beneficial effect on age-dependent muscle fiber denervation, muscle fiber loss, and skeletal muscle atrophy.

In addition to the transgenic non-human animal overexpressing NT, the present invention provides a method for producing said animals, transgenic constructs useful for overexpression of NT, cell lines over expressing NT, as well as methods for screening modulators of NT to alleviate, prevent or delay the progression of sarcopenia.

Now, in order to implement these and still further objects of the invention, which will become more readily apparent as the description proceeds, the following definitions apply:

As used herein, the term “transgene” refers to a nucleic acid sequence introduced into the cells of a non-human animal or into a cell line by way of human intervention.

The term “heterologous” is used herein to describe a sequence which is introduced into a cell derived from a different animal species or cell line different to the one it is introduced in.

The term “homozygous” describes a diploid organism which carries two alleles of a particular transgene.

The term “hemizygous” or “heterozygous” refers to a diploid organism which carries one allele of a particular transgene.

The transgenic non-human animal comprises a transgene encoding neurotrypsin (NT) and overexpresses NT.

The non-human animal of the present invention may be a mammal, for example a rodent such as a mouse or a rat, or a lagomorph such as a rabbit. However, the invention also encompasses pigs, sheep and non-human primates.

Advantageously, the transgene encodes a heterologous NT, i.e. the NT stems form a species which is different to the species into which the transgene is introduced. In one embodiment, the transgene encodes human NT (see SEQ ID No. 2 disclosed in US 2005-0032694 A1). For example, a transgenic mouse may express human NT. For specific applications, it may also be desirable to-overexpress homologous NT, i.e. a transgenic mouse as described herein overexpressing murine NT (see SEQ ID No. 4 disclosed in US 2005-0032694 A1).

The non-human animal described herein may be either hemizygous or homozygous for the transgene, i.e. carrying copies of the transgenic construct encoding NT of either one or both parents, respectively. Preferably, the animal is hemizygous Further, the transgenic animal may carry more than two copies of the transgenic neurotrypsin.

The phenotype of the transgenic non-human animal exhibits symptoms of sarcopenia. When compared to wildtype animals, the transgenic non-human animals show muscle weakness, particularly a reduced total number of muscle fibers (especially in the soleus and EDL) and an inhomogeneous size of said fibers. The motility of said animals is thus reduced. Further, the endplates on sternomastoid, diaphragm and soleus muscles show earlier and more pronounced fragmentation when compared to a wildtype animal. The fiber type distribution of soleus is changed. Further, a mild fiber type grouping is observed as well as an accumulation of internal nuclei in muscle fibers.

In another aspect, the present invention discloses a cell line comprising a transgene encoding NT and overexpressing NT. The transgene may either encode a heterologous or homologous NT. Further, the cell line may be either homozygous or hemizygous for the transgene.

The cell lines disclosed herein may be produced by a method comprising the steps of:
(a) providing a transgenic construct comprising the coding sequence of neurotrypsin;
(b) transforming suitable host cells with the transgenic construct; and
(c) selecting transformants which overexpress neurotrypsin.

Suitable cell lines for the overexpression include, without being limited to, COS cells, CHO cells, HeLa cells, H9 cells, Jurkat cells, NIH3T3 cells, C127 cells, CV1 cells, myeloma cells (such as J558), HEK cells and insect cells. In addition, the expression of neurotrypsin is also possible in yeast expression systems.

In one embodiment, the transgenic construct is the vector described below.

The methods for transformation and evaluating the presence of the introduced nucleic acid as well as its expression are readily available and well-known in the art. Such methods include, without being limited to, hybridization techniques to detect exogenous DNA, PCR, polyacrylamide gel electrophoresis (PAGE) and Western blots to detect DNA, RNA and protein. Further, immunological and histochemical techniques are available to detect NT and the pathology associated with its overexpression.

The present invention further provides a vector comprising a NT coding sequence under the control of a promoter. Said vector may be used for the production of the transgenic non-human animal as disclosed herein or the above described cell line.

The promoter is preferably specific for expression in neurons, such as Thy1. The promoter may also be an inducible promoter or development-specific or tissue-specific.

The NT coding sequence is either heterologous or homologous for the host in which the vector will be transformed. Preferably, the NT coding sequence is human or murine. The vector may further comprise more than one coding sequence of neurotrypsin.

In on particular embodiment, the vector comprises a transcriptional stop cassette flanked by loxP recombination sites and which is inserted between the promoter and the neurotrypsin coding sequence.

The vector typically comprises other elements, such as suitable selection markers and may e.g. be a plasmid vector or based on a suitable virus.

The above described transgenic non-human mammal overexpressing neurotrypsin may be produced by a method comprising the steps of:
(a) preparing an embryo of said non-human mammal wherein the embryo comprises a transgene comprising a nucleotide sequence operably linked to a promoter and encoding a heterologous NT polypeptide;
(b) preparing a pseudopregnant non-human mammal;
(c) implanting the embryo into the pseudopregnant non-human mammal;
(d) allowing said embryo to develop into a live born offspring;
(e) selecting an offspring whose genome comprises said transgene; and
(f) screening the offspring for NT expression.

The embryo of step a) may be generated in different ways well known in the art. One possibility consists in transforming embryonic stem cells with a transgene as described herein and selecting for successfully transformed cells. The selection process may be performed by a number of standard techniques readily available, such as RT-PCR or immunotechiques. Said transformed cells are then injected into the inner cell mass of blastocysts of said mammal. A second possibility is the pronucleus method, wherein the transgene is injected into the male pronucleus of a fertilized egg of a mammal before the pronucleus of the sperm fuses with the pronucleus of the egg. When the prenuclei have fused to form the diploid zygote nucleus, the zygote is allowed to mitotically divide into a two-cell embryo.

The embryos are implanted into a pseudopregnant female. Pseudopregnancy may follow infertile copulation, i.e. by mating a female with a vasectomized male, thereby eliciting hormonal changes which make the uterus of the female receptive. A percentage of the implanted embryos will develop into a live born offspring which has to be tested for the presence of the desired gene. Positive individuals are hemizygous for the transgene.

Optionally, conditional overexpression is desired, i.e. the transgene is inserted into the genome in an inactive form. In said particular embodiment, the transgenic construct may harbor a transcriptional stop cassette preventing the transcription of the neurotrypsin cDNA. Said cassette may be inserted between the promoter and the neurotrypsin coding sequence. In individuals harboring only the promoter-stop-neurotrypsin construct, no neurotrypsin is expressed, as the transcriptional stop cassette prevents the transcription of the neurotrypsin cDNA. Induction of NT expression is achieved by crossing the positively selected offspring with a non-human mammal of the same species expressing cre-recombinase. For said purpose, the above described method further includes the step of: (g) crossing the selected offspring of step (e) with a cre-recombinase expressing non-human mammal of the same species as the non-human mammal of step (b).

In the presence of co-expressed cre-recombinase, the stop cassette is excised and the transcription and further expression of neurotrypsin is enabled (see FIG. 1). Depending on the type of promoter, cre-recombinase will be expressed at specific timepoints or in a specific subset of tissues. The CMV promoter drives early embryonic and general expression of the Cre recombinase and, thus, loxP-dependent recombination occurs in all cell types and tissues. In the case of mice, the general deleter strain CMV-cre (Schwenk et al., 1995 A cre-transgenic mouse strain for the ubiquitous deletion of loxP-flanked gene segments including deletion in germ cells. Nucleic Acids Res 23:5080-1) may be used. The resulting double transgenic mice will constitutively express NT. Such recombinations are usually somatic and must be repeated for every generation. It is to be understood that the present invention also encompasses non-human animals, wherein the germline is stably transformed and thus a line can be established that constitutively overexpresses neurotrypsin.

When crossing the mice of step e) with the general deleter strain CMV-cre, where cre-recombinase is expressed under the control of the strong, early, ubiquitous CMV promoter, offspring of double transgenic animals also expressed neurotrypsin without being crossed to any cre-mouse.

Animals with a relatively weak constitutive-overexpression of NT are viable and can be established in a line by inbreeding the hemizygous animals of step f).

In mice, the weak phenotype is characterized by a lower weight than wild type mice and a somewhat reduced motility. On the diaphragm, the neurofilaments show a “brush” phenotype as the nerve fibers grow out from the central nerve in a similar way as in agrin knock-out mice (FIG. 8). The endplates on sternomastoid, diaphragm, and soleus muscles are fragmented earlier and more pronounced then in wild type mice (FIG. 9). There are also less endplates on the diaphragm. The total number of muscle fibers is reduced by approximately 30% in e.g. the soleus muscle at 3 month age, and the individual fibers show very inhomogeneous sizes (FIG. 10). This muscle phenotype is very similar to the muscle phenotypes observed in human sarcopenia.

Through the described method, non-human mammals being hemizygous for the transgene may be generated. It is also possible to generate homozygous animals, by adding the following steps to the above described method:
(h) crossing two individuals of the offspring of step (g); and
(i) screening the offspring of (h) for individuals being homozygous for the transgene.

Homozygous individuals show a more severe phenotype. Non-human animals bearing the neurotrypsin cassette on both alleles will overexpress double the amount of neurotrypsin than normal hemizygous animals. This leads to a more severe muscle weakness, formation of a hunchback, strongly impaired mobility and reduced fertility.

The pharmacological tuning of NT's activity provides an unprecedented access to the regulatory machinery of the neuromuscular junction and the maintenance of muscle mass and strength. Experiments suggest that inhibiting NT's proteolytic activity, hence shifting the synaptic balance towards synapse-stabilizing activities, reverses synaptic deterioration at the neuromuscular interface, and protects muscles from structural and functional degeneration. A potential therapeutic compound for the treatment, delay of progression, prevention, of sarcopenia may e.g. be detected by measuring its capacity to block the physiological effect of NT or to block the expression of NT.

Hence, a further aspect of the invention is a method for screening a candidate compound for its efficacy in alleviating, preventing or delaying the onset and/or progression of symptoms of sarcopenia. The method comprising the steps of:
(a) administering the candidate compound to a first transgenic non-human mammal as described herein;
(b) determining the performance and/or histological read-out of said mammal; and
(c) comparing the results of step (b) with the performance and/or histological read-out of a second transgenic mammal to which the compound has not been administered; wherein an improved performance of the first mammal compared to that of the second mammal indicates efficacy of the compound.

Different test methods for determining the performance and/or histological read-out are possible. “Performance” describes in the scope of the present invention physiological read-outs such as, without being limited to, muscle strengths or endurance. Histological read-outs include e.g. the number of muscle fibers, the heterogeneity of fiber size, the degree of fragmentation of the endplates and infiltration of the connective tissue.

Another aspect of the invention is a method for screening a candidate compound for its efficacy in alleviating, preventing or delaying the onset and/or progression of sarcopenia. For said method, non-human transgenic animals as disclosed herein are used where the method comprises the steps of:
(a) administering the candidate compound to a first transgenic non-human mammal as disclosed herein prior to the appearance of a selected sarcopenia-related phenotypic trait in said mammal; and
(b) comparing the age at which said selected sarcopenia-related phenotypic trait appears in said mammal with the age at which said trait appears in a second transgenic mammal to which the compound had not been administered; wherein an increased age of appearance of the trait in the first mammal compared to that in the second mammal indicates efficacy of the compound.

It is to be understood that in both methods, the first and second mammal have the same genotype.

Further, the compounds identified by the described methods will be formulated in accordance to known methods to produce pharmaceutically acceptable compositions for the treatment, delay of progression, prevention, of sarcopenia. Said compositions may be administered to a subject in need thereof in a variety of standard ways.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1 schematically shows transgenic constructs and the generation of neurotrypsin-overexpressing mice.

FIG. 2 shows the quantification of the number of synapses per volume of tissue in the neuropil of the stratum radiatum of the CA1 region of the hippocampus. In all experimental animals, the number of synapses per volume of tissue was determined from electron microscopic sections taken from the same location in the stratum radiatum of the CA1 region of the hippocampus wt: wild type; CMV-Cre: transgenic line expressing the Cre recombinase under the control of the CMV promoter; 491 (inact.Nt): transgenic line 491, bearing the inactive transgene, containing a transcriptional stop segment; 494 (inact.Nt): transgenic line 494, bearing the inactive transgene, containing a transcriptional stop segment; DTG(Nt491/cre): double transgenic mouse descending from the line 491, in which-the inactive neurotrypsin transgene has been activated by crossing in the Cre recombinase; DTG(Nt494/cre): double transgenic mouse descending from the line 494, in which the inactive neurotrypsin transgene has been activated by crossing in the Cre recombinase. (**, p<0.01).

FIG. 3 shows electron microscopic comparisons of synapses of the hippocampal stratum radiatum of wild-type and neurotrypsin overexpressing mice. Synapses of neurotrypsin overexpressing mice are smaller than synapses of wild-type mice. Note also the relatively small number of synaptic vesicles in the presynaptic axon terminals of neurotrypsin-overexpressing mice.

FIG. 4 shows the quantification of the axon terminal area in the neuropil of the stratum radiatum of the CA1 region of the hippocampus. In all experimental animals, the axon terminal area of axons that form asymmetric synapses was determined from electron microscopic sections taken from the same location in the stratum radiatum of the CA1 region of the hippocampus wt: wild type; CMV-Cre: transgenic line expressing the Cre recombinase under the control of the CMV promoter; 491 (inact.Nt): transgenic line 491, bearing the inactive transgene, containing a transcriptional stop segment; 494 (inact.Nt): transgenic line 494, bearing the inactive transgene, containing a transcriptional stop segment; DTG(Nt491/cre): double transgenic mouse descending from the line 491, in which the inactive neurotrypsin transgene has been activated by crossing in the Cre recombinase; DTG(Nt494/cre): double transgenic mouse descending from the line 494, in which the inactive neurotrypsin transgene has been activated by crossing in the Cre recombinase. (*, p<0.05; * p<0.01).

FIG. 5 shows the quantification of the synaptic lengths of axospinous synapses in the neuropil of the stratum radiatum of the CA1 region of the hippocampus. In all experimental animals, the number of synapses per volume of tissue was determined from electron microscopic sections taken from the same location in the stratum radiatum of the CA1 region of the hippocampus. As a measure of the synaptic length, the length of the parallel alignment of the presynaptic and the postsynaptic membrane enclosing the synaptic cleft was measured wt: wild type; CMV-Cre: transgenic line expressing the Cre recombinase under the control of the CMV promoter; 491 (inact. Nt): transgenic line 491, bearing the inactive transgene, containing a transcriptional stop segment; 494 (inact.Nt): transgenic line 494, bearing the inactive transgene, containing a transcriptional stop segment; DTG(Nt491/cre): double transgenic mouse descending from the line 491, in which the inactive neurotrypsin transgene has been activated by crossing in the Cre recombinase; DTG(Nt494/cre): double transgenic mouse descending from the line 494, in which the inactive neurotrypsin transgene has been activated by crossing in the Cre recombinase. (*, p<0.05).

FIG. 6 shows the quantification of the cross-sectional area of dendritic spines in the neuropil of the stratum radiatum of the CA1 region of the hippocampus. In all experimental animals, the postsynaptic dendritic spines that form synapses were determined from electron microscopic sections taken from the same location in the stratum radiatum of the CA1 region of the hippocampus wt: wild type; CMV-Cre: transgenic line expressing the Cre recombinase under the control of the CMV promoter; 491 (inact. Nt): transgenic line 491, bearing the inactive transgene, containing a transcriptional stop segment; 494 (inact.Nt): transgenic line 494, bearing the inactive transgene, containing a transcriptional stop segment; DTG(Nt491/cre): double transgenic mouse descending from the line 491, in which the inactive neurotrypsin transgene has been activated by crossing in the Cre recombinase; DTG(Nt494/cre): double transgenic mouse descending from the line 494, in which the inactive neurotrypsin transgene has been activated by crossing in the Cre recombinase. (*, p<0.05).

FIG. 7 shows the spines on secondary dendritic branches of CA1 pyramidal neurons of wild-type mice (A and B) and double-transgenic mice overexpressing neurotrypsin (C and D). CA1 pyramidal cells were iontophoretically filled with biocytin during in vitro electrophysiological studies and visualized using avidin-biotin-peroxidase histochemistry. Dendrites of wild-type mice have many long, well-developed spines (large arrows); in addition, many short, stubby-shape spines (small arrowheads) are also found. Dendrites of neurotrypsin-overexpressing mice (littermates) are dominated by short stubby-shape spines (small arrowheads); long, well-developed spines (large arrows) are very rare. Note, also, that the total spine density (number of spines per unit length of dendrite) is markedly lower in neurotrypsin-overexpressing mice (C and D).

FIG. 8 shows neurofilament brushes in diaphragm muscle of a wildtype (left) and a NT overexpressing mouse (M491S; right).

FIG. 9 shows endplate morphology in diaphragm muscle of a wildtype (left) and a NT overexpressing mouse (M491S; right).

FIG. 10 shows the reduced fiber number in soleus muscle at 3 months age of wildtype NT (left; 907 fibers) and NT overexpressing mouse (M491S; right; 609 fibers).

### MODES OF CARRYING OUT THE INVENTION

### Example 1

### Production of Neurotrypsin

Neurotrypsin is a secreted multi-domain protein with a length of 875 amino acids and an estimated size of 97 kDa for human neurotrypsin and 761 amino acids and a size of 85 kDa for mouse neurotrypsin. The expression of this serine protease as an active protein is dependent upon proper folding and very likely on post-translational modifications, e.g. N-glycosylation which has been proposed for 2 sites in the case of the human and 3 sites for the mouse protein (Gschwend, T. P. et al., Mol. Cell. Neurosci. 9, 207-219, 1997; Proba K., et al., Biochim. Biophys. Acta 1396, 143-147, 1998). In addition, neurotrypsin contains a signal peptide directing the protein to the endoplasmic reticulum from where it is secreted. Neurotrypsin is not an integral membrane protein since it is lacking a transmembrane domain as determined by a hydrophobicity plot by Kyte and Doolittle (Kyte, J. and Doolittle, R. F., J. Mol. Biol. 157, 105-132, 1982). The zymogen activation site of neurotrypsin shows high similarity to the one of tPA (tissue-type plasminogen activator; Tate, K. M. et al., Biochemistry 26, 338-343, 1987). Cleavage at this site by a protease leads to the two fragments, one containing the non-catalytic domains with an apparent molecular weight of 55 kDa (for mouse neurotrypsin) or 67 kDa (for human neurotrypsin) and one containing only the protease domain with 30 kDa. This two-chain form is still linked by a disulfide bond.

Because proper folding and secretion of proteins depends on many, not yet fully understood cellular and molecular mechanisms, several eukaryotic expression systems have been tested for optimal production and secretion of neurotrypsin, including baculovirus-mediated expression in insect cells, stable expression in mouse myeloma cells, and transient expression in human embryonic kidney cells (HEK). These systems have the advantage that they can easily be adapted to serum-free conditions to reduce the amount of contaminating proteins in the supernatant and to set-ups for large-scale production.

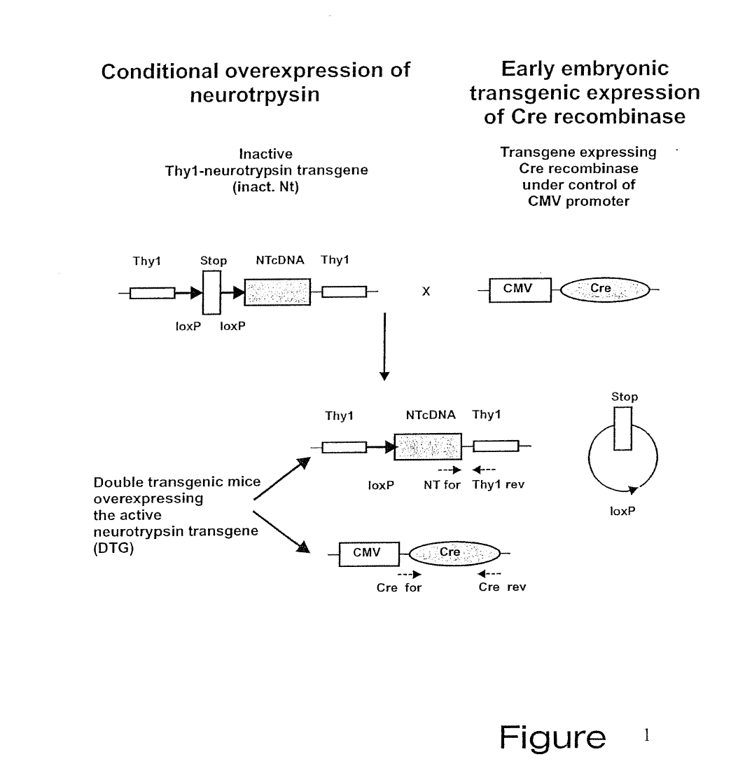

FIG. 1 shows on the upper left the construct for the conditional overexpression of neurotrypsin. In this conditional transgene, the coding part of the neurotrypsin cDNA was incorporated into the Thy1 gene and, thus, put under the control of the Thy1 promoter. In the graph, the first box marked by Thy1 indicates the transcription-regulating promoter at the 5′ end of the Thy-1 gene. The second box marked by Thy1 indicates the 3′ terminal sequences of the Thy1 gene. Between the Thy-1 promoter and the cDNA encoding neurotrypsin, a stop codon flanked by two loxP sequences was inserted. Transcription from the Thy-1 promoter of this transgene, thus, stops before reaching the coding sequence of neurotrypsin. The inactive transgene can be converted into an active transgene by Cre recombinase. Cre recombinase promotes recombination at the loxP sites and, thus, excision of the transcriptional stop sequence. On the upper right side, the construct for expression of Cre-recombinase under the control of the cytomegalovirus (CMV) promoter is shown. Therefore, activation of the inactive transgene can be obtained by crossing the mice containing the inactive Thy1-neurotrypsin (inact.Nt) transgene with the mice containing the CMV-Cre transgene. If a heterozygous inact.Nt mouse is crossed with a heterozygous CMV-Cre mouse, the double-transgenic mice among the offspring express the Thy1-neurotrypsin transgene in the activated form. The expressed Cre-recombinase deletes the transcriptional stop sequence by promoting recombination at the loxP sequences. One loxP sequence remains within the activated Thy1-neurotrypsin transgene. The removed segment composed of the other loxP sequence and the transcriptional stop sequence is shown on the right. The mice were genotyped with the PCR method. The dashed arrows mark the region of the oligonucleotide primers used in the PCR.

### Example 2

### Overexpression of Neurotrypsin in Neurons Using Transgenic Mice Technology.

The overexpression of a gene in a transgenic mouse is used widely to study the function of a protein in vivo. For the first series of experiments, neurotrypsin was overexpressed under the control of the promoter of the Thy-1 gene. The Thy-1 gene is expressed in the nervous system of the mouse relatively late (postnatal day 4-10, depending on the location). Therefore, the expression of neurotrypsin under the control of the Thy-1 promoter (Gordon J. W. et al., Cell 50: 445-452, 1987) ensures that the earlier developmental stages are not perturbed by the presence of excessive amounts of neurotrypsin. This point is essential. Neurotrypsin is expressed in some regions of the developing nervous system relatively early and, thus, it could play a role in early developmental functions, such as cell migration, axon outgrowth, and synapse formation (Wolfer, D. P. et al., Molec. Cell. Neurosci. 18: 407433, 2001). By using a late onset promoter, we intended to prevent perturbations of early stages of neurogenesis in the transgenic animals. However, depending on the aim of an investigation, other promoters may be used as well.

The construct of the first generation transgene was based on an expression vector for Thy-1 in which the translated region of Thy-1 had been substituted by a Xhol linker (Gordon, J. W. et al., Cell 50: 445-452, 1987). The full-length cDNA of neurotrypsin was inserted into the Thy-1 expression vector at the Xhol linker site by a blunt-end ligation and the orientation controlled by means of a restriction enzyme digestion and nucleotide sequence analysis. The plasmid is rescued and the fragment to be used for the injection into the pronucleus of fertilized mouse oocytes was cut out by digestion at the two flanking Pvul sites. The injection fragment was separated by electrophoresis in a 1% agarose gel, the band purified with a QIAEXII-kit, and the DNA eluted from the QIAEX particles with injection buffer. The generation of transgenic mice was achieved by pronuclear injection following standard protocol. The litters were screened for the presence of the transgene by polymerase chain reaction (PCR) and Southern blotting.

We found that transgenic mice that overexpressed the neurotrypsin protein in CNS neurons died shortly after birth. To overcome this problem, we generated a second generation of transgenic mice. These mice bore a conditional transgene that was inactive as long as it had not been activated. To generate an inactive, but activatable, transgene, a removable transcriptional stop sequence was introduced before the neurotrypsin-cDNA. This sequence causes transcription to come to a halt. To make the stop sequence removable, an approach based on the Cre/loxP recombination system was chosen (Sauer B. et al., Proc. Natl. Acad. Sci. (USA) 85: 5166-5170, 1988). The Cre (Cre-recombinase) protein is encoded by the Escherichia coli bacteriophage P1, and efficiently promotes both intra- and intermolecular recombination of DNA in E. coli. Recombination occurs at a specific site called loxP (Hamilton, D. L. and Abremski, K., J. Mol. Biol. 178: 481-486, 1984). This characteristic feature of the Cre recombinase allows deletion and insertion of specifically denoted strings of DNA between the loxP sequences. It can be used to generate specific functional mutations in vivo. This construct was then inserted between the regulatory subunits of the Thy-1 gene (Chen S. et al., Cell 51: 7-19, 1987).

Transfected heterozygous mice with this gene construct were crossed with heterozygous mice carrying Cre-recombinase DNA attached to a cytomegalovirus (CMV) promoter to receive double transgenic (neurotrypsin-overexpressing) mice (see FIG. 1). This promoter is continuously active in vivo and the expressed Cre-recombinase promotes recombination at the two loxP sequences. This procedure removes the transcriptional stop sequence from the inactive transgene and allows transcription of the neurotrypsin cDNA. The transgenic mice were genotyped by PCR and Southern blot hybridization. The DNA for the PCR was extracted from the tail of the mice.

The position of the PCR primers was chosen so that the detection of the native murine neurotrypsin gene was prevented. The 3′-primer corresponded to a DNA sequence inside Thy-1.2 and the 5′-primer to a sequence inside the neurotrypsin cDNA. This DNA fragment is unique to the neurotrypsin transgene. The primers for detection of the Cre insert were both equivalent to DNA sequences derived from inside the Cre gene, because Cre usually does not exist in mice. By this procedure, three mouse lines overexpressing the human neurotrypsin and four lines overexpressing the mouse neurotrypsin were raised. The expression of the transgene was verified at the mRNA level by Northern blotting and in situ hybridization and at the protein level by Western blotting. A typical overexpression was in the order of 2- to 3-fold.

In order to control for the dependence of the neurotrypsin-mediated alterations on the catalytic function of neurotrypsin, transgenic mice overexpressing an inactive form of neurotrypsin under the same (Thy-1) promoter were generated. Inactive neurotrypsin can readily be generated by mutating the essential active site serine 711 to an alanine. Because in all serine proteases, the active site serine is involved in a covalent intermediate of the proteolytic reaction, its mutation results in a complete loss of catalytic function. The transgenic mice overexpressing the inactive form of neurotrypsin were healthy and did not exhibit any abnormalities.

By the same method, transgenic animals expressing full-length neurotrypsin, as well as other truncated forms of neurotrypsin or mutated forms of neurotrypsin (point mutations or deletion mutations) may be generated. Instead of the Thy-1 promoter, other promoters may be used, including promoters driving transgene expression in particular subpopulations of neurons, such as the promoter of the Purkinje cell-specific L7 protein or the promoter of the limbic system-specific protease neuropsin. Alternatively, transgene expression may be put under the control of inducible promoters.

### Example 3

### Overexpression of Neurotrypsin in Neurons of Transgenic Mice Results in a Reduced Number of Synapses in the Cerebral Cortex and the Hippocampus

Excessive amounts of neurotrypsin cause a significant change in number and morphology of the synapses in the central nervous system. Evidence for structural changes was found both with electrophysiological and morphological methods. A physiological correlate of a reduced neuronal surface area in neurotrypsin-overexpressing mice was found with electrical recordings of the capacitance. The time-course of the change of capacitance after a voltage step revealed a significantly reduced whole-cell capacitance in hippocampal neurons of neurotrypsin-overexpressing mice. A reduced whole cell capacitance may be due to a reduced surface area of the membrane, if other possible causes can be excluded. Importantly, changed membrane transductance was excluded experimentally as a cause of the observed change. Therefore, we have investigated the extension and the branching pattern of the neuronal dendrites as well as the size of the neuronal somas. We found no significant deviation of the size at the branching pattern of the neuronal dendrites between neurotrypsin-overexpressing and wild-type mice. Neuronal somas were, if anything, rather enlarged. The observation of a reduced whole-cell capacitance without a reduction of the surface area of the cell soma and the dendrites strongly suggests the reduction in the dendritic spines. The dendritic spines contribute between 50% and 70% to the surface of the neuron and are not included in the measurements of the length and branching pattern of the dendrites. This possibility was evaluated by counting and measuring synapses in neuropil regions and inspecting dendritic spines along dye-filled dendrites.

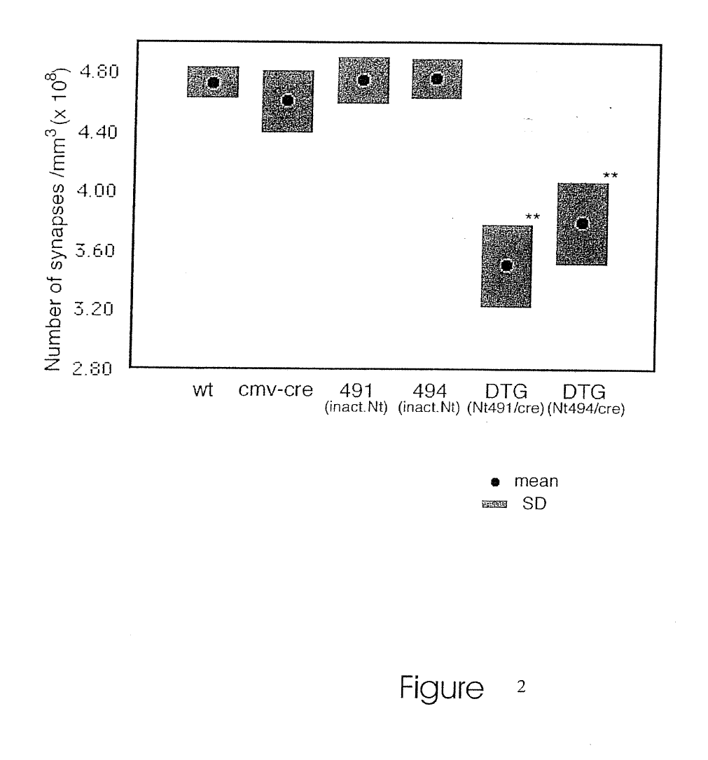

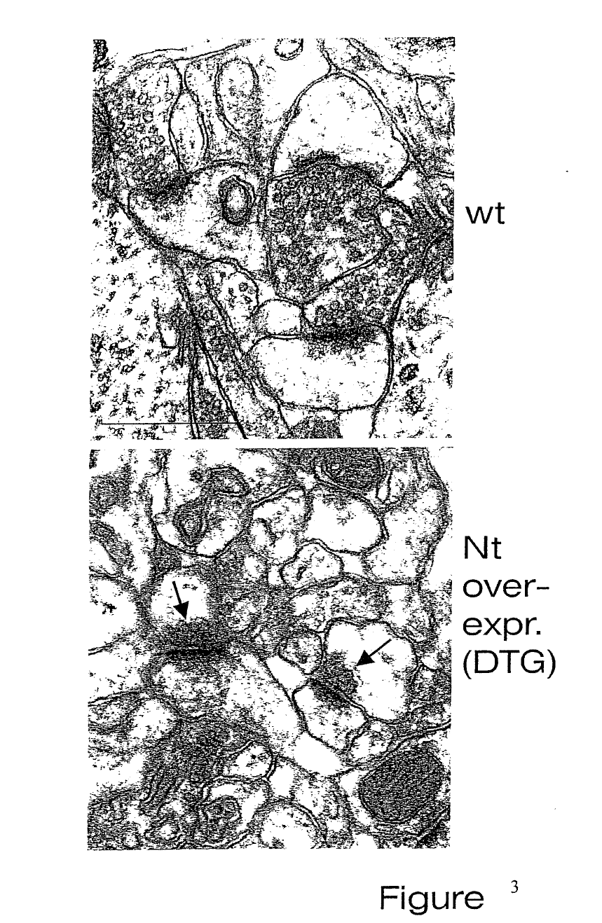

We found a reduction, both in the number of total synapses per area (FIG. 2) and in three measurements reflecting synaptic size, namely the area of the presynaptic axon terminal, the area of the postsynaptic spine, and the synaptic length (determined as the length of the apposition of the presynaptic and the postsynaptic membranes. By inspection of the spines along dye-filled dendrites, we found a reduction in the size and the number of spines in the neurotrypsin-overexpressing mice (FIG. 3). These measurements are in mutual agreement, because many synapses end on dendritic spines. Therefore, fewer synapses and fewer dendritic spines represent two readouts of the same phenomenon. In addition, reductions in the size of the presynaptic terminal, the postsynaptic spines, and other synaptic parameters, such as synaptic length were found in accordance with smaller, less-well developed spines observed after dye-labeling of dendrites.

### Example 4

### Increased Levels of Neurotrypsin in CNS Neurons Result in Reduced Number and Size of Synapses (Results Revealed by Quantitative Morphology)

In this experiment, we attempted to quantify the number of synapses per volume of tissue of a synapse-rich region, and to measure the size parameters of the synapses. Parameters measured included the area of the presynaptic axon terminals, the area of the postsynaptic spines, and the length of the synapses (as measured by the length of the apposition of the pre- and postsynaptic membrane). Two independent lines of neurotrypsin-overexpressing mice (Nt491/cre and Nt494/cre) and several lines of control mice (wildtype mice, CMV-Cre mice, and the transgenic parental lines bearing the inactive neurotrypsin transgene (Nt491-inact.Nt and Nt494-inact.Nt) were investigated.

The mice were deeply anesthetized at the age of 28 days with metofane (Schering-Plough, USA) and perfused through the heart with 0.9% sodium chloride followed by fixative consisting of 2% paraformaldehyde, and 1% glutaraldehyde in 0.1 M phosphate buffer pH 7.4 (PB). The brains were removed from the skull and sectioned into 100 μm-thick serial sections with a vibratome. The sections were postfixed in 1% osmium tetroxide in PB, treated with 2% uranyl acetate, dehydrated in ethanol and propylene oxide and embedded in Durcupan ACM resin (Fluka). For electron microscopic analysis strips of sections containing the CA1 region of the hippocampus at the anteriocaudal level Bregma-2 mm and mediolaterally 1.5 mm were ultrasectioned. An illustration of the EM images obtained is given in FIG. 2.

The synaptic sampling procedure consisted of 15 to 23 EM samples of the neuropil of the stratum radiatum of the hippocampal CA1 region from three noncontiguous areas with at least 50 μm distance between each other an initial magnification of 27,500-fold. The electron micrographs were printed at a final magnification of 80,000-fold which represented 90 to 135 μm² of tissue. A synapse was defined as two apposed thickened membranes of a presynaptic and postsynaptic profile, with the presynaptic profile containing at least three synaptic vesicles in close association with the differentiated membranes. The synapses were classified into axodendritic and axospinous synapses according to ultrastructural criteria. Dendritic shafts were identified by their size and the presence of mitochondria and microtubules. Dendritic spines were of smaller diameter, lacked mitochondria and microtubules, and occasionally contained a spine apparatus. The axodendritic synapses comprised an insignificantly small proportion in all samples and therefore were excluded from further statistical estimation. All axospinous synapses were counted in each micrograph with exception of those touching the exclusion lines (an unbiased counting frame, Gundersen, H. J. G., J. Microsc. 111: 219-223, 1977). The cross-section areas of axonal terminals and postsynaptic spines and lengths of synaptic junctions of all axospinous synapses were measured directly from the prints using a magnetic tablet (Kurta) and the Macstereology 2.8 (Ranfurly Microsystems, UK) analysis program. The numerical density of synapses were obtained using size-frequency method and formula N_{V}=N_{A}/d (were N_{A} is a number of synaptic profiles per unit area and d is the average length of synaptic junctions; Colonnier, M. and Beaulieu, C., J. Comp. Neurol. 231: 175-179,1985; DeFelipe, J., et al., Cereb. Cortex 9:722-732, 1999).

The number of synapses per cubic mm (mm³) was significantly reduced in neurotrypsin-overexpressing mice (FIG. 2). In contrast, the numbers of synapses in control mice, i.e., the parental lines used for the generation of the double transgenic (DTG) neurotrypsin-overexpressing mice (491-inact.Nt, 494-inact-Nt, and CMV-Cre) were the same as in wild-type mice. Therefore, these results indicate a significant reduction of synapses in the neurotrypsin-overexpressing mice.

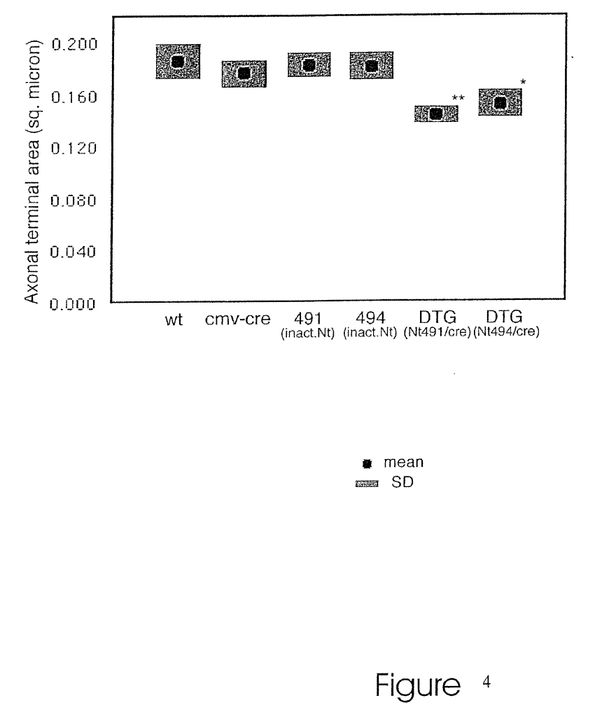

Axonal terminal area was significantly reduced in neurotrypsin-overexpressing mice (FIG. 4). In contrast, the values in control mice, i.e., the parental lines used for the generation of the double transgenic (DTG) neurotrypsin-overexpressing mice (491-inact.Nt, 494-inact-Nt, and CMV-Cre) were the same as in wild-type mice. Therefore, these results indicate a significant reduction of the presynaptic terminal size in the neurotrypsin-overexpressing mice.

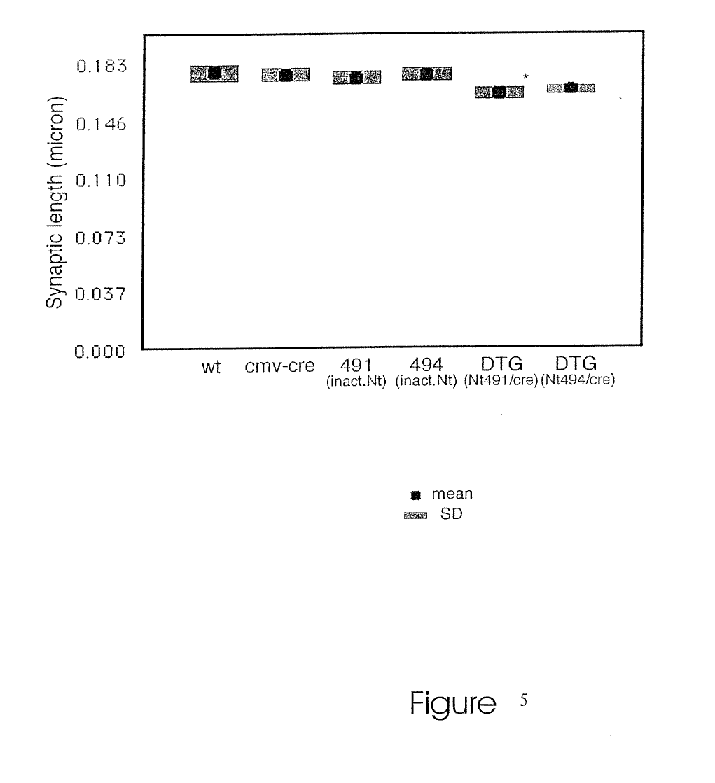

Synaptic length was significantly reduced in neurotrypsin-overexpressing mice (FIG. 5). In contrast, the values in control mice (i.e., the parental lines used for the generation of the double transgenic (DTG) neurotrypsin-overexpressing mice (491-inact.Nt, 494-inact-Nt, and CMV-Cre)) were the same as in wild-type mice. Therefore, these results indicate a significant reduction of the synaptic area in the neurotrypsin-overexpressing mice.

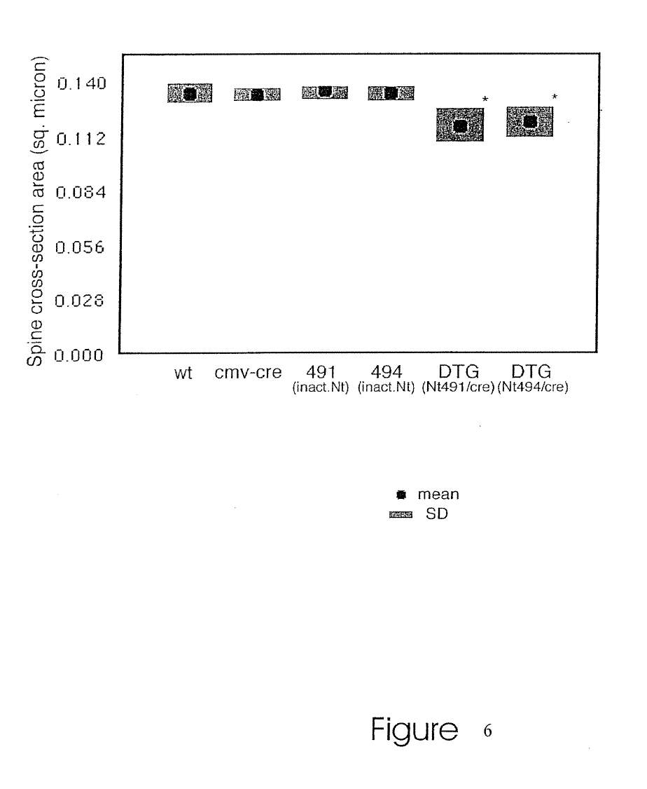

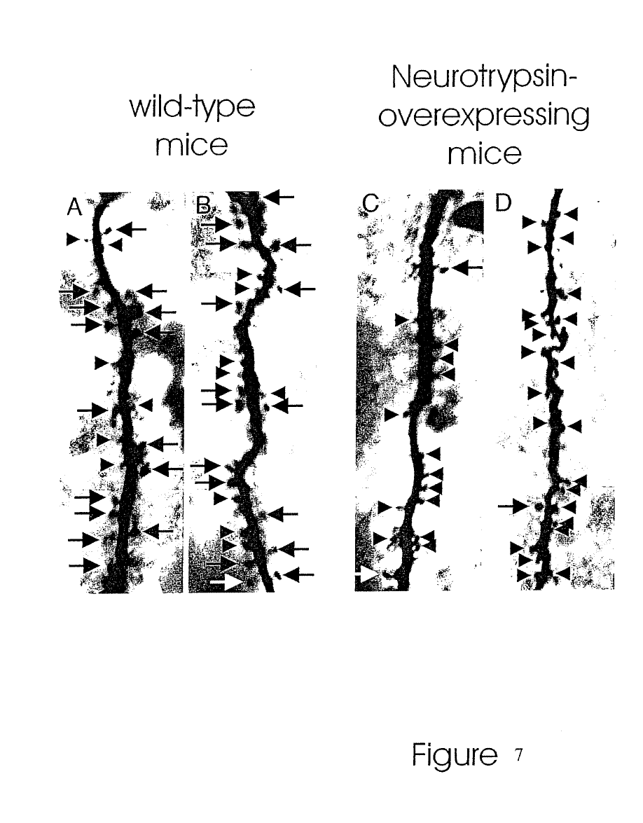

The cross sectional area of the postsynaptic spines was significantly reduced in neurotrypsin-overexpressing mice (FIG. 6). In contrast, the values in control mice, i.e. the parental lines used for the generation of the double transgenic (DTG) neurotrypsin-overexpressing mice (491-inact.Nt, 494-inact-Nt, and CMV-Cre) were the same as in wild-type mice. Therefore, these results indicate a significant size reduction of the postsynaptic spines in the neurotrypsin-overexpressing mice.

In summary, the synapse density, as determined by the number of synapses per mm³, is significantly reduced in the neurotrypsin-overexpressing mice as compared with wildtype and control littermates (FIG. 2). The presynaptic terminals are reduced in size (FIG. 4). The synaptic length, as determined by the length of the apposition of the pre- and postsynaptic membrane, is significantly reduced in neurotrypsin-overexpressing mice (FIG. 5). Similarly, the size of the postsynaptic spines is significantly reduced in neurotrypsin-overexpressing mice (FIG. 6).

In transgenic mice overexpressing the catalytically inactive mutation of neurotrypsin, engineered by mutating the reactive site serine 711 to an alanine (Ser711Ala mutation), these synaptic alterations were not found. In conclusion, the observations made in transgenic mice overexpressing the wild-type form of neurotrypsin are mediated by the proteolytic activity of neurotrypsin.

### Example 5

The diaphragm muscles of 3 months old mice are removed and fixed with methanol for 10 minutes at −20° C. The fixed diaphragms are incubated with anti-neurofilament antibody for 18 hours, washed, and incubated with a fluorescent secondary antibody. After another wash-step, the muscles are mounted on a glass slide with fluorescent mounting medium. Microscopic pictures are taken with a 5× objective.

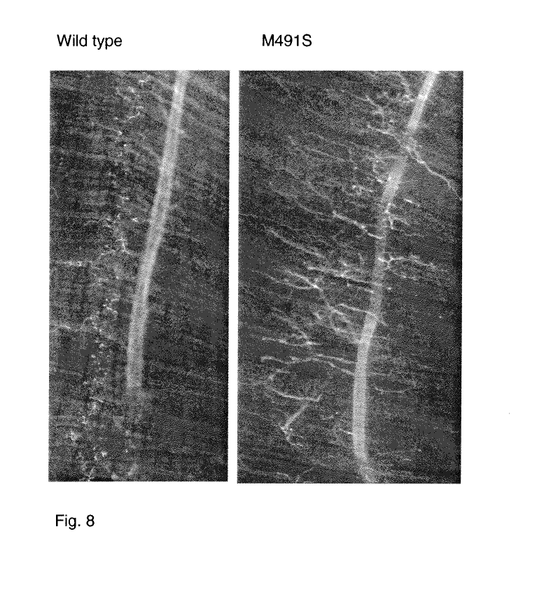

In FIG. 8, sections of mouse diaphragms are depicted. The main nerve is visible in the centre of the picture. Nerve fibers grow out towards the individual endplates (not visible). This outgrowth is much more pronounced in the M491S animals than in wild type animals.

### Example 6

The diaphragm muscles of 3 months old mice are removed and fixed with methanol for 10 minutes at −20° C. The fixed diaphragms are incubated with alpha-bungarotoxin (labeled with fluorescent dye Alexa-488) for 4 hours and washed. Then the muscles are mounted on a glass slide with fluorescent mounting medium. Microscopic pictures are taken with a 40 objective on a Leica LSCM Confocal Microscope. 10-20 sections of 1 μm each are recorded and assembled to confocal stack images.

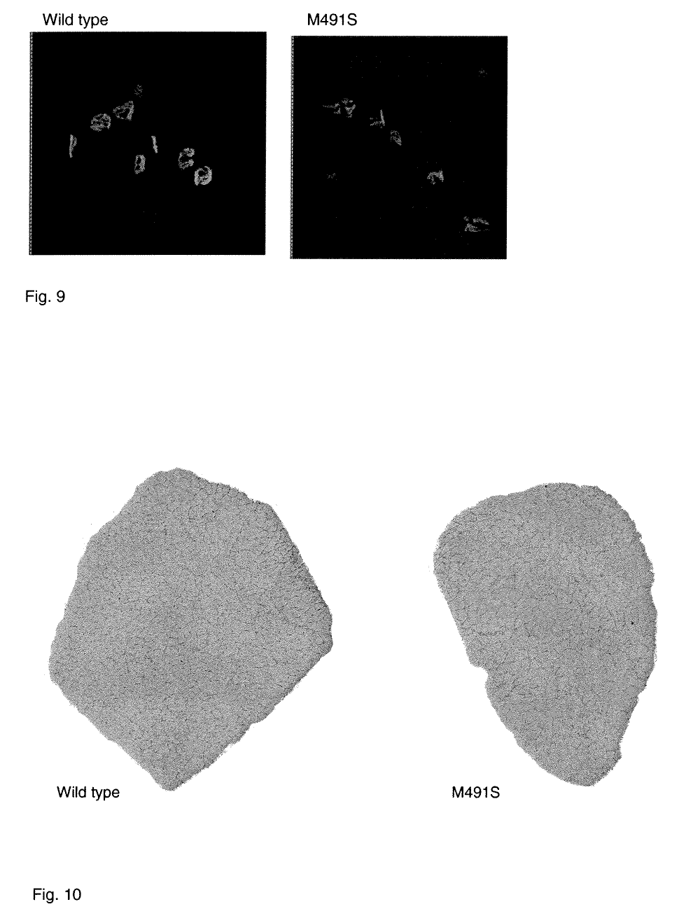

Depicted in FIG. 9 are confocal image stack of mouse diaphragms stained with fluorescent bungarotoxin. This staining reveals the acetylcholine receptors which form the neuromuscular endplate. Wild type endplates show a typical pretzel form. M491S animals have fragmented endplates. This fragmentation eventually leads to the disappearance of individual endplates and therefore to an overall smaller number of endplates.

### Example 7

Soleus muscles are removed from the lower extremities of 3 month old mice, embedded in Tissue-Tek mounting medium, and immediately frozen in isopentane at −85° C. for 10 minutes. Samples are the stored in a −80° C. freezer. Frozen muscles are cross-sectioned in a Cryotome at −25° C. into 12 μm sections. Cryo-sections are mounted on Superfrost glass slides and stained with eosin/hematoxylin. Stained samples are sealed with Eukitt and a cover slip and stored at 4° C.

Depicted are microscopic pictures (1.5× magnification) of 12 μm cryo-sections of mouse soleus muscles of 3 month old mice stained with eosin/hematoxylin. The Neurotrypsin-overexpressing animal has a reduced fiber number (about 30%) and an increased inhomogeneity of fiber sizes.

While there are shown and described presently preferred embodiments of the invention, it is to be distinctly understood that the invention is not limited thereto but may be otherwise variously embodied and practiced within the scope of the following claims.

1. A transgenic non-human animal comprising a transgene encoding neurotrypsin wherein the animal exhibits symptoms of sarcopenia.

2. The non-human animal of claim 1 which is a mammal.

3. The non-human animal of claim 1 which is a rodent.

4. The non-human animal of claim 1 which is a mouse.

5. The non-human animal of claim 1 wherein the neurotrypsin is heterologous.

6. The non-human animal of claim 5 wherein the neurotrypsin is human neurotrypsin.

7. The non-human animal of claim 1, being heterozygous for the transgene.

8. A cell line comprising a transgene encoding neurotrypsin and overexpressing neurotrypsin.

9. A vector comprising a neurotrypsin coding sequence under the control of a promoter.

10. The vector of claim 9, wherein the neurotrypsin coding sequence is human.

11. The vector of claim 9, wherein the promoter is specific for expression in neurons, in particular Thy1.

12. The vector of claim 9, wherein a transcriptional stop cassette flanked by loxP recombination sites is inserted between the promoter and the neurotrypsin coding sequence.

13. A method for producing a transgenic non-human mammal overexpressing neurotrypsin comprising the steps of:
(a) preparing an embryo of said non-human mammal wherein the embryo comprises a transgene comprising a nucleotide sequence operably linked to a promoter and encoding a heterologous neurotrypsin polypeptide;
(b) preparing a pseudopregnant non-human mammal;
(c) implanting the embryo into the pseudopregnant non-human mammal;
(d) allowing said embryo to develop into a live born offspring;
(e) selecting an offspring whose genome comprises said transgene; and
(f) screening the offspring for neurotrypsin expression.

14. The method of claim 13, further comprising between steps (e) and (e the step of (g) crossing the selected offspring of step (e) with a cre-recombinase expressing non-human mammal of the same species as the non-human mammal of step (b).

15. The method of claim 14, wherein the method additionally comprises the step of (h) crossing two individuals of the offspring of step (g); and (i) screening the offspring of (h) for individuals being homozygous for the transgene.

16. The method of claim 13, wherein heterologous neurotrypsin is human neurotrypsin.

17. The method of claim 13, wherein the non-human mammal is a rodent.

18. The method of claim 13, wherein the non-human mammal is a mouse.

19. A method for screening a candidate compound for its efficacy in alleviating, preventing or delaying the onset and/or progression of symptoms of sarcopenia, the method comprising the steps of:
(a) administering the candidate compound to a first transgenic non-human mammal of claim 2;
(b) determining the performance and/or histological read-outs of said mammal; and
(c) comparing the performance and/or histological read-outs of said mammal with the performance and/or histological read-outs of a second transgenic mammal of the same type to which the compound has not been administered; wherein an improved performance of the first mammal compared to that of the second mammal indicates efficacy of the compound.

20. A method for screening a candidate compound for its efficacy in preventing or delaying the onset and/or progression of sarcopenia, the method comprising the steps of:
(a) administering the candidate compound to a first transgenic non-human mammal of claim 2 prior to the appearance of a selected sarcopenia-related phenotypic trait in said mammal; and
(b) comparing the age at which said selected sarcopenia-related phenotypic trait appears in said mammal with the age at which said trait appears in a second transgenic mammal to which the compound had not been administered; wherein an increased age of appearance of the trait in the first mammal compared to that in the second mammal indicates efficacy of the compound.
